(19) United States
(12) Patent Application Publication
(10) Pub. No.: US 2006/0212045 A1
(43) Pub. Date: Sep. 21, 2006

(54) Surgical suture cutter

(76) Inventors: Peter Schilling, Tuttlingen (DE); Lothar U. Hipp, Tuttlingen-Nendingen (DE); James A. Rinner, Franksville, WI (US); Darius P. Daruwala, West Harrison, NY (US)

Correspondence Address: BAKER & HOSTETLER LLP, WASHINGTON SQUARE, SUITE 1100, 1050 CONNECTICUT AVE. N.W., WASHINGTON, DC, 20036-5304, US

(21) Appl. No.: 11/370,187

(22) Filed: Mar. 8, 2006

Related U.S. Application Data
(60) Provisional application No. 60/659,457, filed on Mar. 8, 2005.

(51) Int. Cl. A61B 17/10

(52) U.S. Cl. 606/138

(57) ABSTRACT

A suture cutter having a handle and a barrel with a movable cutting head on the barrel, and the barrel supporting a stationary cutting edge and a movable cutting edge. A movable support carries the movable cutting edge and includes a surface spaced from the stationary cutting edge a selected distance. An abutment wall spaced from the stationary cutting edge at the selected distance provides for snugly restricting movement of the moving cutting edge away from cutting contact with the stationary cutting edge while cutting the suture. Recesses are defined on a guiding support surface along which the movable cutting edge slides when the handle is actuated, allowing for sharpening of the moveable cutting edge when said edge is retracted proximally.

### CROSS-REFERENCE TO RELATED APPLICATIONS

This application claims priority from U.S. provisional patent application No. 60/659,457, filed Mar. 8, 2005, the disclosure of which is hereby incorporated by reference in its entirety.

### FIELD OF THE INVENTION

This relates to medical devices, and, more particularly, to a surgical suture cutter which has a handle portion and a barrel portion.

### BACKGROUND OF THE INVENTION

The prior art is already aware of cutters with squeezable handles which support a cutter blade for operating the cutter blade in cutting operation of a line to be cut. Those cutters also have provision for interchanging the cutting portion with respect to the supporting handle for selection of particular cutters to be applied to the line to be cut. However, many of the prior art cutters suffer from imprecise cutting elements, resulting in cutting actions that can be unstable or difficult to perform during surgery. Furthermore, the cutters are difficult to clean and do not provide a reliable means of cleaning or sharpening the cutting blades.

It is desirable therefore to provide a device that solves the deficiencies of the prior art cutters in that, among other features, it provides an enhanced accuracy of cutting, an enhanced stability and ease of cutting, as well as providing for interchangeability of the cutting head and an attachment or coupling to the suture to be cut.

Sutures are of abrasive and otherwise tough material. It is desirable therefore to provide a cutter which will both readily cut the suture and will be arranged for continuously presenting a sharp cutting edge to the many sutures it is required to cut, and do so in a facile manner for the demanding environment of surgery. It is further desirable for the cutter to be arranged to be readily attached to the suture to be cut, and to then be accurately positioned along a length located on the suture where the cut is desired.

Further, it is desirable that the cutting edge of the cutter be readily sharpened, and the entire cutting head be easily replaced relative to the handle, whereby it can then be securely connected to the handle.

### SUMMARY OF THE INVENTION

The foregoing needs are met, to a great extent, by the present invention, wherein in one aspect an apparatus is provided that in some embodiments provides a surgical suture cutting instrument that provides an enhanced accuracy of cutting, an enhanced stability and ease of cutting, and provides for interchangeability of the cutting head and an attachment or coupling to the suture to be cut. The cutting edge of the instrument is configured and shaped so that it can be readily sharpened, while the entire cutting head can be easily replaced relative to the handle, whereby it can then be securely connected to the handle.

In accordance with one embodiment of the present invention, a medical device for cutting sutures is provided, including a handle member coupled to a barrel having a longitudinal axis spanning the axial direction of the barrel. A sliding cutting member is disposed at the distal end portion of the barrel and coupled through the barrel to the handle portion so as to slide axially along the longitudinal axis by actuation of the handle member. The sliding cutting member defines a first cutting edge disposed on a distal head of the sliding cutting member. The distal end portion of the barrel further defines a second cutting edge parallel to the first cutting edge and a guiding surface disposed opposite the second cutting edge by a receiving width substantially equal to a width of the distal head of the cutting member, the distal head being insertable through the receiving width by actuation of the sliding cutting member by the handle member.

In accordance with another aspect of the present invention, a suture cutting medical instrument is provided, which includes a handle portion and a barrel portion having a longitudinal axis. A sliding block is included with a moveable cutting edge movable relative to and past a stationary cutting edge fixed on a distal end portion of said barrel portion. The distal end portion of said barrel portion defines a first guiding surface facing said stationary cutting edge and being spaced therefrom a first distance in a direction transverse to the longitudinal axis. The barrel portion supports the moveable cutting edge disposed on said sliding block, where a distal end portion of said sliding block having a width substantially equal to the first distance less a sliding tolerance margin, such that when a suture is disposed between said moveable cutting edge and said stationary cutting edge, it is simultaneously pressed upon by both said cutting edges in the direction transverse to the longitudinal axis.

In accordance with yet another embodiment aspect of the present invention, a suture cutting medical device is provided, having a handle member coupled to a barrel having a longitudinal axis spanning the axial direction of the barrel. A sliding cutting member is disposed at the distal end portion of the barrel and coupled with an actuation means to the handle portion so as to slide axially along the longitudinal axis by actuation of the handle member. The sliding cutting member has a first means for cutting a suture disposed on a distal head of the sliding cutting member. The distal end portion of the barrel further defines a second means for cutting a suture disposed parallel to the first means. And a guiding means is disposed opposite the second means by a width substantially equal to a width of the distal head of the sliding cutting member, the guiding means stabilizing the sliding cutting member to engage the first and second means to cut a suture.

There has thus been outlined, rather broadly, certain embodiments of the invention in order that the detailed description thereof herein may be better understood, and in order that the present contribution to the art may be better appreciated. There are, of course, additional embodiments of the invention that will be described below and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of embodiments in addition to those described and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

FIG. 1 is a side elevation view of a suture cutter of this invention, with an intermediate portion broken away.

FIG. 2 is a top plane view of FIG. 1.

FIG. 3 is a section view taken on a plane designated by the line 3-3 on FIG. 2.

FIG. 4 is an enlarged perspective view of a front end portion of FIG. 1.

FIG. 5 is a rear perspective view of FIG. 4.

FIG. 6 is a top perspective view similar to FIG. 4 but with a part removed.

FIG. 7 is a top plan view of FIG. 6.

FIG. 8 is a front elevation view of FIG. 7.

FIGS. 9 and 10 are respectively right end and left end elevation views of FIG. 8.

FIG. 11 is a bottom plan view of FIG. 8.

FIGS. 12 and 13 respectively are a bottom perspective view and a side elevation view of a cutter part in FIG. 4.

FIGS. 14 and 15 respectively are left end and right end elevation views of FIG. 13.

FIGS. 16 and 17 respectively are a bottom plan view and a top plan view of FIG. 13.

FIG. 18 is an enlarged top perspective view, with a suture captured therein, of the right end of FIG. 4, but turned around from the showing in FIG. 4.

FIGS. 19 and 20 are top perspective views similar to FIG. 18, but with the suture progressively further captured therein.

FIG. 21 is a top perspective view similar to FIG. 18 but with the cutter now beyond the cutting action, and showing the suture already cut.

FIG. 22 is a section view taken on a plane designated by the line 22-22 of FIG. 5.

### DETAILED DESCRIPTION

In accordance with conventional practice, as used herein, the term “proximal” or “proximal end” shall refer to the specified end of a device or its component which is generally closer to the medical personnel handling or manipulating the device as it is intended to be used, and the term “distal” or “distal end” shall refer to the specified end of a device or its component which is opposite the proximal end.

Generally the drawings show a suture cutter having a handle portion 10 and a barrel portion 11. The distal end of the barrel has a hook 12 which snares the suture to be cut, and the hook can then be slid along the suture to a selected length location for making the actual cut as desired. The handle includes a support portion 13 which has a bolt 14 passing therethrough, and a nut 16 secures the bolt to the handle. The bolt 14 has a passageway 17 therethrough, and the barrel 11 is nested therein for secure mounting of the barrel. Also, the barrel 11 and the handle portion 13 have tongue-and-groove connections therebetween at 18 to affix the barrel axially of itself on the handle. With that bolted arrangement, the barrel can be replaced relative to the handle, as desired. It should be understood that other connection means can be employed for replaceably mounting the barrel onto the handle and to extend along the longitudinal axis of the barrel.

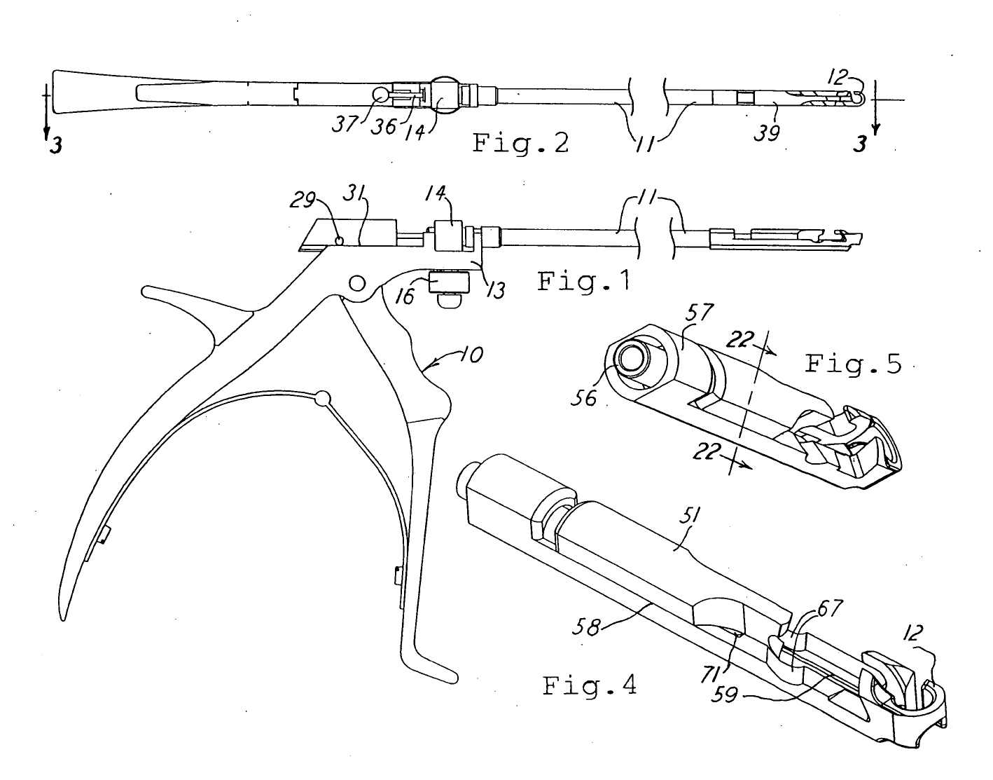

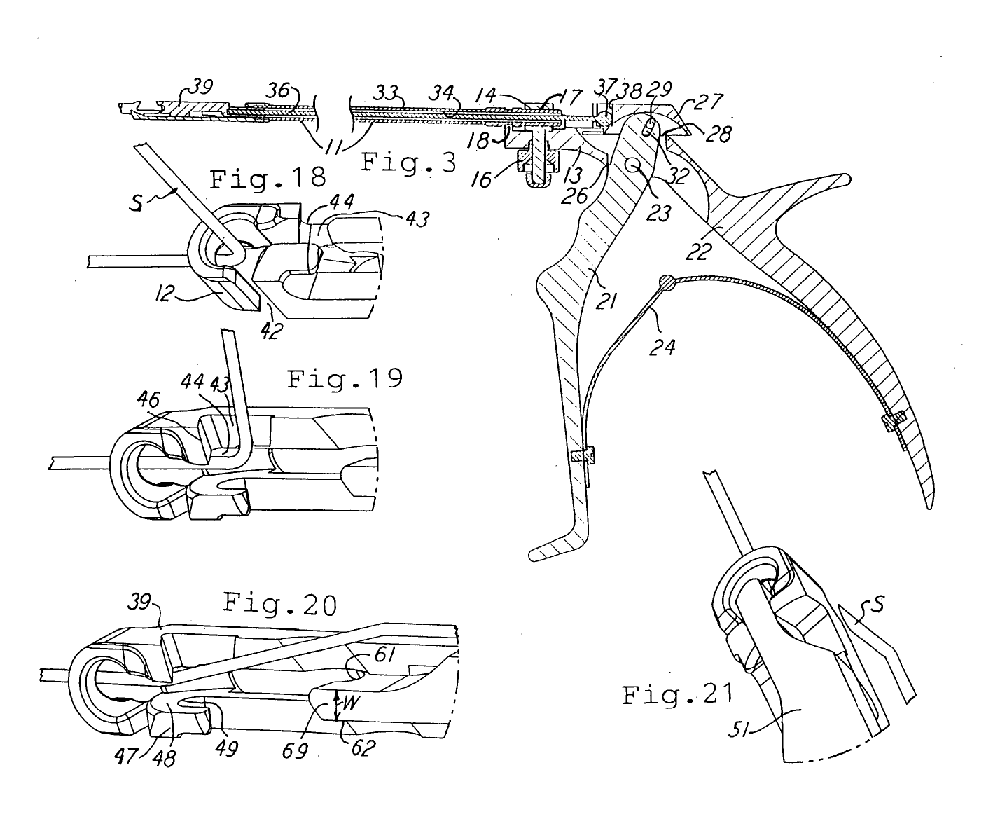

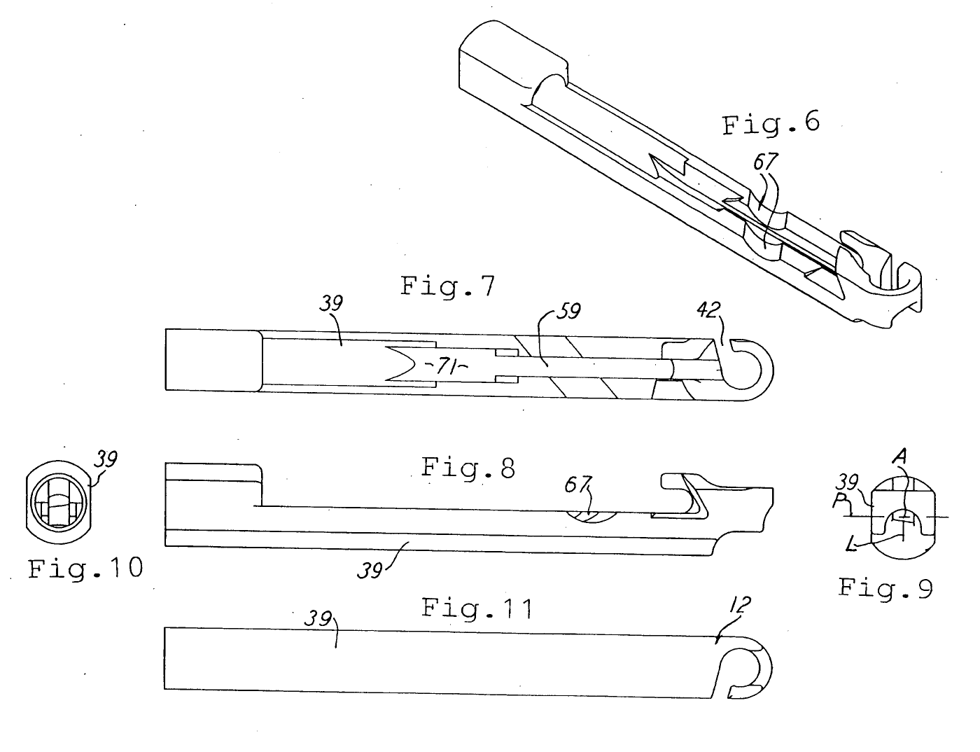

The handle portion 10 has two grips 21 and 22 which are pivotally connected together by a pin 23, and the grips are urged apart by an interposed spring 24 and will pivot apart until the grip 21 abuts grip 22 at stop surface 26. A sliding block 27 is on the top of the grip 22 at surface 28 disposed at the end of grip portion 21, and there can be an upwardly shouldered guide for restricting the block 27 from rising off the surface 28, such as indicated. A pin 29 is affixed to the block 27 and it rides on a surface 31 on the top end of the handle grip portion 22, as seen in FIG. 1. Also, the handle 21 has a slot 32 which receives the pin 29 for moving the pin as the handle is actuated, and thus also moving the block 27 left and right, as viewed in FIGS. 1 and 3. It may be considered that the grip 21 pivots counterclockwise in cutting operation, and relative to the grip 22.

The barrel 11 has a tube 33 which presents a hollow bore 34. A rod 36 is axially slideable in the tube 33, and it is connected to the block 27 by a ball 37 trapped in an opening 38 in the block 27. The distal end of the tube 33 has a guide member 39 fixedly attached thereto which includes at its distal end the suture hook 12, which also defines a suture entry opening 42 on a first side, for attaching to a suture S, as shown in FIG. 18. The distal end of guide member 39 also defines a second opening 43 on a second side opposite to the aforementioned first side for the exit stringing of the suture S, as seen in FIG. 19. Further, the member 39 defines a suture passageway 44 extending between the entry opening 42 and the exit opening 43. A suture stationary and straight cutting edge 46 is also between the entry 42 and the exit 43 and it is presented to be opposed to the suture S. A slide guide 47 is on the member 39, and it has a planar face 48 and a curved edge 49 on the face 48.

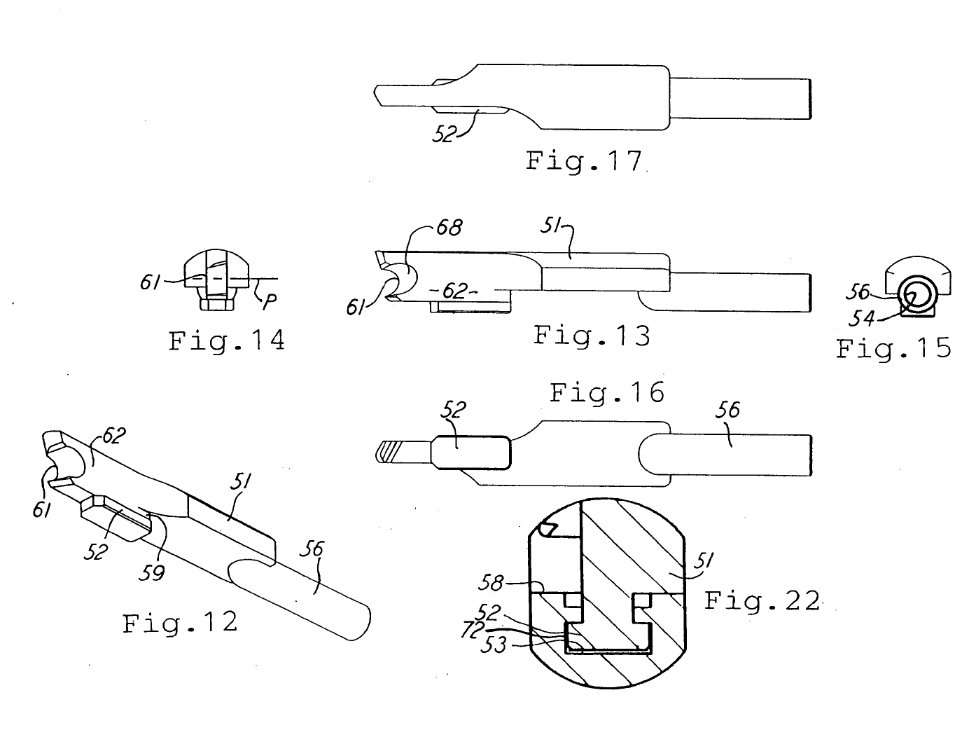

FIGS. 12-17 show a sliding cutter member 51 which is nested with the member 39, such as by a T-connection shown in FIG. 22, so the member 51 can not deviate laterally relative to its supporting member 39. That is, member 51 is slideably mounted on member 39 and can have a T-flange 52 disposed snugly within a T-slot 53 defined by the member 39 and along a portion of its distal end, as shown. The member 51 can have a cylindrical opening 54 to effect a telescopic connection with the rod 36, as seen in FIGS. 3 and 15, to thereby be axially fixedly connected with the rod 36 and move therewith upon squeezing actuation of the handle portion 10. A cylindrical end 56 on the member 51 is slideably guided in the barrel end 57 at the proximal end portion of guiding member 39. Also, the members 39 and 51 have planar surfaces intersecting along plane 58 for sliding contact and guidance, and there can be sliding surfaces in the form of a tongue-and-groove sliding contact created by tongue element 59 disposed in groove 59 defined within guiding member 39.

The sliding cutting member 51 is thus snugly guided in sliding contact relative to the member 39 as its moves along the longitudinal axis A aligned with the axis of tube 33, said axis A shown in FIG. 9 oriented perpendicular to the plane of the figure as defined by axes P and L.

The member 51 has an arcuate shaped cutting edge 61 facing the cutting edge 46. Upon movement of the member 51 by squeezing the handles 21 and 22, the member 51 will advance to the suture S and force it between the two cutting edges 46 and 61 to sever the suture as indicated in FIG. 21. In that cutting action, the stationary member 39 presents its slide surface 48 which engages a planar surface 62 on the member 51. The surface 48 faces the cutting edge 46, and the moving cutting member 51 can not laterally deviate from snug contact between the cutting edges 46 and 61. So the suture S is accurately and cleanly and easily severed by the arcuate cutting edge 61 sliding into contact and over the straight cutting edge 46. With the concavity of edge 61, the suture is trapped by the cutting edges for the desired cutting action.

For sharpening the cutting edge 61 while the instrument is still assembled as shown, two recesses 67 are in the member 39 for ready access to and contact on the edge 61 by an unshown file or like sharpener. Thus the member 39 is positioned to have the cutting edge 61 adjacent to and held at the recesses 67. The cutting edge 61 is terminal to the arcuate surface 68 which is angled relative to the longitudinal axis of the instrument, as seen in FIG. 13. Likewise, the recesses are offset from each other in the longitudinal instrument direction, as seen in FIG. 4, so they align with the cutting face 68. The two recesses permit sharpening action from either side of the instrument.

In a preferred embodiment, the distance between the abutment wall 48 and the cutting edge 46 is substantially the same as the width W of the head 69 which supports the moving cutting edge 61. Thus the surface 48 restricts transverse movement of the cutting edge 61 away from the cutting edge 46 during cutting action, and the width of the portion 48 is only very slightly less, within sliding tolerance, than the distance between the two cutting edges when they are in their cutting positions. Surfaces 48 and 62, and thus cutting edges 46 and 61, are in sliding contact with each other during cutting action because the surfaces serve as lateral abutments and guide the cutting head 69 toward the cutting edge 46.

FIG. 9 shows a longitudinal plane L which presents a longitudinal axis A along which the cutter member 51 slides, and from which it will not deviate, while being laterally guided and restricted, such as by the T-connection of FIG. 22. The cutting edge 46 and abutment surfaces 48 and 62 are simultaneously parallel to the plane L perpendicular to the plane P during cutting action. Cutting edge 61 is concave relative to cutting edge 46, so the suture is trapped between the two cutting edges.

For assembly, member 51 can have its proximal end portion 56 passed into the sleeve 57 and then moved to where the tongue 52 is placed into a wide groove 71 defined by member 39. Then the member 51 can be lowered and slid to where the tongue 51 enters the narrower T-groove 53. The tongue 52 is on member 51 near the same end thereof as the location of the cutting edge 61. The T-connection thus renders lateral support for the distal portion end of the member 51 and for the cutting edge 61 to present cutting edge 61 into shearing contact with cooperating shear cutting edge 46. The tongue 52 and groove 53 have mutually sliding surfaces at 72 in FIG. 22 to effect the sliding contact between the cutting edges 46 and 61.

Further producing that sliding contact between the cutting edges is the block 47 which is positioned across suture holding passageway 46 from the cutting edge 46, as seen in FIG. 20. So, while shear cutting, the cutting head 69 is contacted by both the cutting edge 46 and the sliding shoe type guide 48. So the sliding shoe surface 48 directly faces the cutting edge 46, and a perpendicular normal vector line off the face 48 intersects the cutting edge 46.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents maybe resorted to, falling within the scope of the invention.

What is claimed is:

1. A medical device for cutting sutures, comprising:
a handle member coupled to a barrel having a longitudinal axis spanning the axial direction of the barrel,
a sliding cutting member disposed at the distal end portion of the barrel and coupled through the barrel to the handle portion so as to slide axially along the longitudinal axis by actuation of the handle member,
the sliding cutting member defining a first cutting edge disposed on a distal head of the sliding cutting member,
the distal end portion of the barrel further defining a second cutting edge parallel to the first cutting edge and a guiding surface disposed opposite the second cutting edge by a receiving width substantially equal to a width of the distal head of the cutting member, the distal head being insertable through the receiving width by actuation of the sliding cutting member by the handle member.

2. The medical device of claim 1,
wherein the distal end portion of the barrel further defines a hook member for capturing a suture.

3. The medical device of claim 2,
wherein the distal end portion of the barrel further defines a suture passageway for positioning the suture between the first and second cutting edges prior to actuation of the sliding cutting member to engage the suture with the first and second cutting edges to sever the suture, the suture passageway being disposed between the hook member and the sliding cutting member.

4. The medical device for cutting sutures of claim 1,
wherein the sliding cutting member slides upon actuation by the handle member along a groove defined by a guiding member disposed at the distal end portion of the barrel, the sliding cutting member being removable from the groove to be separated from the barrel of the medical device.

5. The medical device for cutting sutures of claim 4,
wherein the guiding member includes two recesses aligned on opposite sides of the sliding cutting member at an axial position on the barrel along the longitudinal axis such that when the sliding cutting member is retracted from the distal end portion of the barrel, the first cutting edge is aligned with the two recesses to permit a sharpening instrument to be inserted therethough to sharpen the first cutting edge.

6. The medical device for cutting sutures of claim 1,
wherein the first cutting edge is defined by a cutting face at the distal end of the sliding cutting member and the cutting face and two recesses are aligned parallel to a plane non-perpendicular to the longitudinal axis.

7. A suture cutting medical instrument, comprising:
a handle portion and a barrel portion having a longitudinal axis, and
a sliding block with a moveable cutting edge movable relative to and past a stationary cutting edge fixed on a distal end portion of said barrel portion, said distal end portion of said barrel portion defining a first guiding surface facing said stationary cutting edge and being spaced therefrom a first distance in a direction transverse to the longitudinal axis, and
said barrel portion supporting the moveable cutting edge disposed on said sliding block, a distal end portion of said sliding block having a width substantially equal to the first distance less a sliding tolerance margin, whereby a suture disposed between said moveable cutting edge and said stationary cutting edge is simultaneously pressed upon by both said cutting edges in the direction transverse to the longitudinal axis.

8. The suture cutting medical instrument of claim 7,
wherein the distal end portion of the barrel portion further defines a hook member for capturing a suture.

9. The suture cutting medical instrument of claim 8,
wherein the distal end portion of the barrel portion further defines a suture passageway for positioning the suture between the moveable cutting edge and stationary cutting edge prior to actuation of the sliding block to engage the suture with the moveable cutting edge and stationary cutting edge to sever the suture, the suture passageway being disposed between the hook member and the sliding block.

10. The suture cutting medical instrument of claim 7,
wherein the sliding block slides upon actuation by the handle portion along a retaining groove defined by a guiding member disposed at the distal end portion of the barrel portion, the sliding block being removable from the retaining groove to be separated from the medical device.

11. The suture cutting medical instrument of claim 10,
wherein the guiding member includes two recesses aligned on opposite sides of the sliding block at an axial position on the barrel portion along the longitudinal axis such that when the sliding block is retracted from the distal end portion of the barrel portion, the moveable cutting edge is aligned with the two recesses to permit a sharpening instrument to be inserted therethough to sharpen the moveable cutting edge.

12. The suture cutting medical instrument of claim 7,
wherein the moveable edge is defined by a cutting face at the distal end of the sliding block and the cutting face and two recesses are aligned parallel to a plane non-perpendicular to the longitudinal axis.

13. The suture cutting medical instrument of claim 7,
wherein the handle portion is actuated by two grip members coupled by a spring element.

14. A suture cutting medical device, comprising:
a handle member coupled to a barrel having a longitudinal axis spanning the axial direction of the barrel,
a sliding cutting member disposed at the distal end portion of the barrel and coupled with an actuation means to the handle portion so as to slide axially along the longitudinal axis by actuation of the handle member,
the sliding cutting member having a first means for cutting a suture disposed on a distal head of the sliding cutting member,
the distal end portion of the barrel further defining a second means for cutting a suture disposed parallel to the first means,
and a guiding means disposed opposite the second means by a width substantially equal to a width of the distal head of the sliding cutting member, the guiding means stabilizing the sliding cutting member to engage the first and second means to cut a suture.

15. The suture cutting medical device of claim 14,
wherein the distal end portion of the barrel further defines a hook means for capturing a suture.

16. The suture cutting medical device of claim 15,
wherein the distal end portion of the barrel further defines a passageway means for positioning the suture between the first means for cutting a suture and second means for cutting a suture prior to actuation of actuation means to engage the sliding cutting member so that the suture is cut between the first and second means, the passageway means being disposed between the hook means and the sliding cutting member.

17. The suture cutting medical instrument of claim 14,
wherein the sliding cutting member slides upon actuation of the actuation means along a retaining groove defined by a guiding member disposed at the distal end portion of the barrel, the sliding cutting member being removable from the retaining groove to be separated from the medical instrument.

18. The suture cutting medical instrument of claim 17,
wherein the guiding member includes two recesses aligned on opposite sides of the cutting member at an axial position on the barrel along the longitudinal axis such that when the sliding cutting member is retracted from the distal end portion of the barrel, a cutting edge of the first cutting means is aligned with the two recesses to permit a sharpening instrument to be inserted therethough to sharpen the cutting edge.

19. The suture cutting medical instrument of claim 14,
wherein the first cutting means includes a cutting face at the distal end of the sliding cutting member and the cutting face and two recesses are aligned parallel to a plane non-perpendicular to the longitudinal axis.

20. The suture cutting medical instrument of claim 14,
wherein the handle member is actuated by two grip members coupled by a spring means.
